(19) United States
(12) Patent Application Publication
(10) Pub. No.: US 2021/0015762 A1
(43) Pub. Date: Jan. 21, 2021

(54) COMPOSITIONS FOR TREATING OBESITY

(71) Applicant: J. Phillip KENNEDY, Grand Rapids, MI (US)

(72) Inventor: J. Phillip KENNEDY, Grand Rapids, MI (US)

(73) Assignee: Truetiva, Inc., Grand Rapids, MI (US)

(21) Appl. No.: 16/930,564

(22) Filed: Jul. 16, 2020

Related U.S. Application Data
(60) Provisional application No. 62/874,798, filed on Jul. 16, 2019.

(51) Int. Cl. A61K 31/05; A61K 47/42; A61K 47/20; A61K 36/87; A61K 36/15; A61K 36/185; A61K 36/48; A61K 36/81; A61K 36/736; A61K 36/73; A61K 36/45; A61P 3/04

(52) U.S. Cl. CPC A61K 31/05; A61K 47/42; A61K 47/20; A61K 36/87; A61K 36/15; A61P 3/04; A61K 36/48; A61K 36/81; A61K 36/736; A61K 36/73; A61K 36/45; A61K 36/185

(57) ABSTRACT

Embodiments of the invention are directed to compositions containing cannabinoids and bioenhancers for treating obesity, eating disorders, metabolic disorders, and other body weight related diseases and disorders, and methods for treating obesity, eating disorders, metabolic disorders, and other body weight related diseases and disorders by administering compositions containing cannabinoid and a bioenhancer.

### CROSS-REFERENCE TO RELATED APPLICATIONS

This application claims priority from U.S. Provisional No. 62/874,798 entitled “Compositions For Treating Obesity,” filed Jul. 16, 2019, the entire contents of which are hereby incorporated by reference.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Obesity is a major public health concern and is now recognized as a chronic disease that requires treatment to reduce its associated health risks. Although weight loss is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Currently available prescription drugs for managing obesity generally reduce weight by inducing satiety or decreasing dietary fat absorption. However, the anti-obesity drugs available commercially provide only modest weight loss. The most successful drug regimens in humans have been combinations of phentermine and fenfluramine or of ephedrine, caffeine and/or aspirin. Each of these combinations have been discontinued due to safety concerns. Although investigations are on-going, there still exists a need for a more effective and safe therapeutic treatment for reducing or preventing weight-gain.

### SUMMARY OF THE INVENTION

Various embodiments are directed to compositions containing a cannabinoid, a bioenhancer, and a pharmaceutically acceptable carrier, excipient, diluent, reagent, or combinations thereof, and methods for treating obesity, eating disorders, metabolic disorders, and other body weight related diseases and disorders by administering such compositions to a subject in need of treatment. In some embodiments, the cannabinoid may have a concentration of about 20% (w/w) to about 1% (w/w) cannabidiol, relative to the total amount of the composition, and in certain embodiments, the cannabinoid may be a cannabidiol, cannabidiol isomer, cannabidiol analog, or combinations thereof. In some embodiments, the bioenhancer may have a concentration of about 0.05% wt to about 20% wt, relative to the total weight of the composition. In particular embodiments, the bioenhancer may be a cyclic peptide, and in some embodiments, the bioenhancer may be piperidine or a piperidine derivative. In some embodiments, the composition may further include an appetite suppressant such as, for example, an extract selected from the group consisting of grapes, pine, cocoa, tamarind, tomato, peanut, almond, apple, cranberry and blueberry. In some embodiments, the amount of appetite suppressant in the compositions may be from about 0.05% to about 20% (wt/wt), relative to the total amount of the composition.

### DESCRIPTION OF THE DRAWINGS

The patent or application file contains at least one drawing executed in color. Copies of this patent or patent application publication with color drawing(s) will be provided by the Office upon request and payment of the necessary fee.

Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all of these specific details. In other instances, well known process operations have not been described in details so as to not unnecessarily obscure the present invention.

FIG. 1 is a comparison of the structure of a PD-L1 dimer bound by BMS-202 (left panel) and cannabidiol (right panel).

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with the terms disease, condition, syndrome, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Obesity is a major public health concern and is now recognized as a chronic disease that requires treatment to reduce its associated health risks. Although weight loss is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Currently available prescription drugs for managing obesity generally reduce weight by inducing satiety or decreasing dietary fat absorption. However, the anti-obesity drugs available commercially provide only modest weight loss. The most successful drug regimens in humans have been combinations of phentermine and fenfluramine or of ephedrine, caffeine and/or aspirin. Each of these combinations have been discontinued due to safety concerns. Although investigations are on-going, there still exists a need for a more effective and safe therapeutic treatment for reducing or preventing weight-gain.

Various embodiments are directed compositions and methods for treating obesity, eating disorders, metabolic disorders, and other body weight related diseases and disorders. The compositions of such embodiments may include a cannabinoid and a bioenhancer. The methods of various embodiments may generally include administering a composition containing a dannabinoid and a bioenhancer in effective amounts to a subject in need of treatment. In particular embodiments, the subject may suffer from obesity, eating disorders, or other body weight related diseases and disorders. In certain embodiments, the biohancer may be a cyclic peptide, such as piperidine.

The cannabinoids of such embodiments include any of a broad class of compounds that are known to interact with cannabinoid receptors, and encompass endocannabinoids (produced naturally in the body by animals), the phytocannabinoids (found in cannabis and some other plants), and synthetic cannabinoids (manufactured artificially). Example cannabinoids include, but are not limited to, tetrahydropyran analogs, such as, Δ⁹-tetrahydrocannabinol, Δ⁸-tetrahydrocannabinol, 6,6,9-trimythel-3-pentyl-6H-dibenzo[b, d]pyran-1-ol, 3-(1,1-dimethylheptyl)-6,6a7,8,10,10a-hexahydro-1-1hydroxy-6,6-dimythel-9H-dibezo[b,d]pyran-9-ol, (−)-(3S,4S)-7-hydroxy-delta-6-tetrahydrocannabinol-1,1-dimethylheptyl, (+)-(3S,4S)-7-hydroxy-A-6-tetrahydrocannabinol, and Δ⁸-tetrahydrocannabinol-11-oic acid, piperidine analogs, such as, (−)-(6S,6aR,9R,10aR)-5,6,6a,7,8,9,10,10a-octahydro-6-methyl-1-3-[(R)-1-methyl-4-phenylbutoxy]-1,9 phenanthridinediol 1-acetate), aminoalkylindole analogs, such as, (R)-(+)-[2,3-dihydro-5-methyl-3-(4-morpholinylm-ethyl)-pyrrolo[1,2,3-de]-1,4-benzoxazin-6-yl]-1-naphthelenyl-methanone, open pyran-ring analogs, such as, 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3′,4′5′,6′-hexahydrobiphen yl, lipophilic alkylamides, such as, dodeca-2E,4E,8Z,10E/Z-tetraenoic-acid-isobutylamide, cannabinoid mimetics, salts, solvates, metabolites, and metabolic precursors of these compounds and combinations thereof. In some embodiments, cannabinoids may be derived plants including hemp, Echinacea purpurea, Echinacea angustifolia, Acmella oleracea, Helichrysum umbraculigerum, Radula marginata, and combinations thereof and oils made from these plants, and in other embodiments, cannabinoids may be manufactured or chemically synthesized.

The compositions of various embodiments can include any number of cannabinoids in various concentrations; however, in certain embodiments, the cannabinoid may be cannabidiol (2-(6-sopropenyl-3-methyl-5-cyclohexen-1-yl)-5-pentyl-1,3-benzenediol). Cannabidiol has 7 double bonds and 30 stereoisomers. Embodiments include compositions containing each stereoisomer individually and compositions containing a combination of these stereoisomers. In particular embodiments, the compositions used in the methods of embodiments and the compositions of embodiments may include high concentrations of cannabidiol. For example, in some embodiments, cannabidiol may be about 30 w/v % to about 100 w/v % of the cannabinoids in the composition, and in other embodiments cannabidiol may be about 50 w/v % to about 100 w/v %, about 75 w/v % to about 100 w/v %, about 80 w/v % to about 100 w/v %, about 90 w/v % to about 100 w/v % of the cannabinoids in the composition.

Cannabidiol can be obtained by cold-pressing industrial hemp with trace amounts of THC. Cannabidiol in this present invention is provided as a natural constituent of hemp oil.

In some embodiments, the cannabinoids in the composition may be cannabidiol analogs. The term “cannabidiol analogs” refers to synthetically produced compounds that are structurally similar, but not structurally identical, to cannabidiol. Various cannabidiol analogs are known in the art and embodiments encompass such cannabidiol analogs. For example, PCT Publication WO2017/132526 and U.S. Pat. No. 6,630,507, which are each hereby incorporated by reference in their entireties, describes various analogs of cannabidiol. In some embodiments, the analogs of cannabidiol may be of general Formula I:

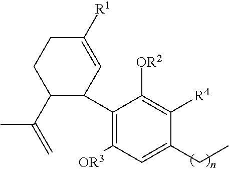

where R¹ is hydrogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are each, individually, hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is hydrogen, substituted or unsubstituted alkyl, carboxyl, alkoxy, aryl, aryloxy, arylalkyl, halo or amino, and n may an integer of 2 to 10 and the like and salts and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof. Like cannabidiol, cannabidiol analogs can have various isomers. Embodiments include all isomers of the such cannabidiol analogs.

In some embodiments, cannabidiol analogs, such as those described above may be combined with cannabidiol, to produce a mixture of cannabidiol and cannabidiol analogs. Thus, as used herein the term “cannabidiol” encompasses cannabidiol, cannabidiol analogs, and the various isomers of cannabidiol and cannabidiol analogs.

The compositions of various embodiments can include up to about 50% (w/w) cannabidiol, cannabidiol analogs, isomers of cannabidiols, and combinations thereof (collectively, “cannabidiol”), relative to the total amount of the composition, and in some embodiments, the compositions may include from about 100% to about 0.5% (w/w) cannabidiol, relative to the total amount of the composition, 50% (w/w) to about 0.5% (w/w) cannabidiol, relative to the total amount of the composition, about 30% (w/w) to about 1% (w/w) cannabidiol, relative to the total amount of the composition, about 20% (w/w) to about 1% (w/w) cannabidiol, relative to the total amount of the composition, about 20% (w/w) to about 5% (w/w) cannabidiol, relative to the total amount of the composition, or any range or individual concentration encompassed by these example ranges. In particular embodiments, the composition may include about 15% (w/w) to about 10% (w/w) cannabidiol, relative to the total amount of the composition, or any range or individual concentration encompassed by this example range.

In certain embodiments, the cannabidiol of embodiments described above may be cannabidolic acid (“CBDA”). Without wishing to be bound by theory, CBDA may exhibit improved hydrophilicity over other isomers of cannabidiol, which may allow for improved solubility and delivery of CBDA to the skin. The CBDA may be modified, partially digested, or otherwise acted upon by enzymes in the skin to produce for example cannabidiol (CBD), which may be the active form cannabidiol in the composition. Thus, CBDA may act as a prodrug in some embodiments of the invention. Other cannabidiol analogs or isomers may produce a similar effect and are encompassed by prodrug embodiments of the invention.

The cannabidiol in the compositions of embodiments of the invention may be 100% cannabidiol, or oils, solvents, and emulsions containing cannabidiol. For example, in some embodiments, the compositions of the invention may include cannabidiol derived from hempseed oil. Hempseed oil is generally manufactured from varieties of Cannabis sativa that do not contain significant amounts of tetrahydrocannabinol (THC), the psychoactive element of the cannabis plant. This manufacturing process typically includes cleaning the seed to 99.99% before pressing the oil. Hempseed oil generally also contains omega-6 and omega-3 fatty acids. For example, about 30-35% of the weight of hempseed oil are essential fatty acids (EFAs), i.e., linoleic acid, omega-6 (LA, 55%), α-linolenic acid, omega-3 (ALA, 22%), γ-linolenic acid, omega-6 (GLA, 1-4%), and stearidonic acid, omega-3 (SDA, 0-2%). Thus, the compositions of some embodiments may contain fatty acids such as omega-6 and omega-3 fatty acids.

Oils include cannabidiol oil and various plant derived oils containing cannabidiol, such as, hempseed oil, Echinacea purpurea, Echinacea angustifolia, Acmella oleracea, Helichrysum umbraculigerum, Radula marginata, and the like. In some embodiments, cannabidiol isolated from such plants or made synthetically may be formulated with an oil such as, for example, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and the like and combinations thereof.

Unless indicated otherwise, the term “therapeutically effective amount” is not particularly limited, so long as the cannabinoid is present in an amount effective for treating the dermatological disease. The therapeutically effective amount of cannabinoid can be from about 2 milligrams per kilogram (mg/kg) to about 100 mg/kg, about 2 mg/kg to about 50 mg/kg, about 2 mg/kg to about 25 mg/kg, or any range or individual concentration encompassed by these example ranges, wherein mg refers to the mass or weight of the cannabinoids and kg refers to the mass or weight of the patient in need of treatment. In certain embodiments, a therapeutically effective amount of THC and/or CBD in the composition may be about 2 mg/kg to about 10 mg/kg or any range or individual concentration encompassed by these example ranges, wherein mg refers to the mass or weight of the cannabinoids and kg refers to the mass or weight of the patient in need of treatment.

Without wishing to be bound by theory, the compositions of various embodiments may block inhibitory checkpoints, reducing inflammation in affected areas upon application. Various proteins have been associated with the immune checkpoint blockade including adenosine A2A receptor (A2AR), B7-H3 or CD276, MGA271, B7-H4 or VTCN1, B and T Lymphocyte Attenuator (BTLA or CD272), Cytotoxic T-Lymphocyte-Associated protein 4 (CTLA-4 or CD152), Indoleamine 2,3-dioxygenase (IDO), tryptophan 2,3-dioxygenase (TDO), Killer-cell Immunoglobulin-like Receptor (KIR), Lymphocyte Activation Gene-3 (LAG3), Programmed Death 1 (PD-1), T-cell Immunoglobulin domain and Mucin domain 3 (TIM-3), V-domain Ig suppressor of T cell activation (VISTA). The cannabidiols of embodiments may block activity of these or other immune checkpoint blockade proteins, reducing immune response and improving the symptoms of dermatitis. For example, the cannabidiols of the compositions of the invention may impact the interaction between transmembrane protein PD-1 and its ligands, PD-1 ligand 1 (PD-L1) and PD-1 ligand 2 (PD-L2) by blocking binding of PD-L1 or PD-L2 with PD-1. Inhibiting the activity of PD-1 may reduce T-cell signaling, preventing the immune response and reducing inflammation in the affected area and reducing the symptoms of dermatitis.

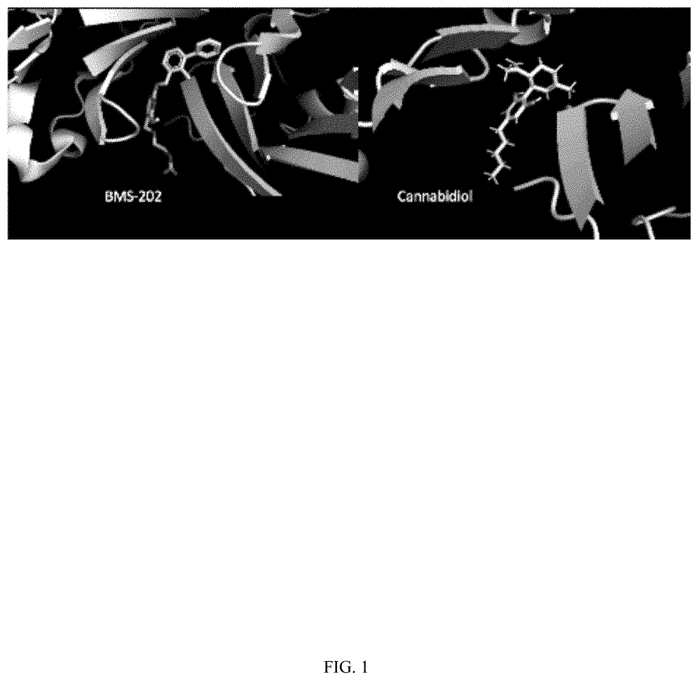

Cannabinoids may bind PD-L1 at a hydrophobic cavity created by dimerization similar to binding of known PD-1 inhibitor BMS-202. FIG. 1 compares models of PD-L1 binding of BMS-202, left panel, and cannabidiol, right panel. Three-dimensional structural similarity and the location of charged moieties suggest a similar binding orientation.

Inhibitory checkpoints like PD-1 and PD-L1 have been linked to cancer. For example, cancer-mediated upregulation of PD-L1 on the cell surface may inhibit T cells that might otherwise attack a tumor or other tumorigenic tissue. Thus, the compositions of various embodiments can be used as anti-cancer agents to block the interaction PD-1 or PD-L1, allowing T-cells to attack the tumors or tumorigenic tissue. In some embodiments, the cancers may be one or more of melanoma, lung cancer, pancreatic cancer, kidney cancer and Hodgkin's lymphoma, for example.

In various embodiments, the cannabidiol containing compositions described above may include one or more additional immune checkpoint blockade inhibitors or the cannabidiol containing compounds of the invention may be administered in combination with one or more additional immune checkpoint blockade inhibitors. Additional checkpoint blockade inhibitors include, for example, an IgG4 PD1 antibody such as, for example, antibody BGB-A317, Nivolumab, or Pembrolizumab, a PD-L1 inhibitor, such as, for example, atezolizumab, avelumab, or and durvalumab, antibodies that block the immune checkpoint molecule CTLA-4 such as, for example, ipilimumab, therapeutic agents that target an intrinsic checkpoint blockade, such as, for example, the gene encoding Cytokine-inducible SH₂-containing protein (CISH). The amount of immune checkpoint blockade inhibitor in the compositions may be from about 0.01% to about 5% (w/w), relative to the total amount of the composition, from about 0.1% to about 1% (w/w), relative to the total amount of the composition, or any range or individual concentration encompassed by these example ranges.

For example, in some embodiments, the compositions may include one or more bioenhancers. Bioenhancers include any compound or composition that aids in the transport of another compound across epithelial membranes. Bioenhancers include P-glycoprotein inhibitors, compounds that reverse P-glycoprotein-mediated efflux, limit metabolism of active agents, increase gastric emptying time and intestinal motility, reduce degradation of the active agent by hydrochloric acid, modify cell membrane permeability, produce a cholagogue effect, modify the bioenergetics and thermogenic properties of the active agent, suppress first pass metabolism, and inhibit metabolizing enzymes, stimulate gamma glutamyl transpeptidase, enhance the uptake of amino acids, and the like and combinations thereof. In some embodiments, the bioenhancers may be herbal or neutraceutical bioenhancers. Examples of bioenhancers encompassed by the invention include piperine, quercetin, genistein, naringin, sinomenine, glycyrrhizin, nitrile glycosides cuminum cyminum, zingiver officinale, lysergol, allium sativum, aloe vera, and the like and combinations thereof. In some embodiments, the bioenhancers may be liposomes, microspheres, nanoparticles, transferosomes, ethosomes, nanoemulsions, microemulsions, lipid based systems, polymeric micelle formulations, ketoprofen-loaded solid lipid nanoparticles, and the like, which can be made from beeswax, carnauba wax, or other natural waxes and solid lipids, and combinations thereof. In some embodiments, the bioenhancers may be liposomal enhancers such as, for example, Ginkgo biloba lipid-based systems, silybin lipid-based systems, ginseng lipid-based systems, hawthorn lipid-based systems, quercetin lipid-based systems, curcumin lipid-based systems, and the like and combinations thereof. In further embodiments, the bioenhancers may be capsaicin transferosomes, colchicine tranferosomes, vincristine tranferosomes, and the like and combinations thereof, which may find particular use as natural skin penetration agents.

In some embodiments, the bioenhancer may be a cyclic amine such as, for example, benzylamines, pyrrolidines, piperidines, morpholines, piperazines, and the like and derivatives thereof. In certain embodiments, the cyclic amine may be piperidine or a piperidine derivative such as N-methylpiperidine, N-ethylpiperidine, N-phenylpiperidine, pelletierine, sedamine, sedridine, allosedridine, dumetorine, solenopsins, isosolenopsins, andrachcinidine, cassine, prosopinine, prosophylline, morusimic acid, and the like and combinations thereof. In some embodiments, the cyclic amine may be isolated from or contained in an extract from a natural source such as pepper, sanguinarine, coptisine, goldenseal, and the like and combinations thereof.

For example, piperine enhances bioavailability by modulating DNA receptor binding and cell signal transduction, while inhibiting efflux pumps that remove the active agent from cells. This inhibits drug metabolizing enzymes and stimulates absorption by stimulating gut amino acid transporters and inhibiting cellular pumps responsible for drug elimination from cells and intestinal production of glucuronic acid. Piperine also increases the absorption of the active agent in the gastrointestinal tract and inhibits enzymes responsible drug metabolism especially in the liver during first pass metabolism such as hepatic arylhydrocarbon hydrolase and UDP-glucuronyltransferase activities. Piperine modifies the rate of glucuronidation by lowering the endogenous UDP-glucuronic acid content and also by inhibiting transferase activity. Piperine inhibits P-glycoprotein and cutochrome P450 3A4, also CYP1A1, CYP1B1, CYP1B2, CYP2E1, CYP3A4, among others and makes target receptors more responsive to drugs, acting as receptors for drug molecules, increasing GIT vasculature by vasodilation to increase the absorption of drugs, modulation of cell membrane dynamics which increases transport of drugs across the cell membranes.

The amount of bioenhancer in the compositions may be from about 0.05% to about 20% (wt/wt), relative to the total amount of the composition or in some embodiments, from about 0.1% to about 10% (wt/wt), relative to the total weight of the composition, from about 0.1% to about 5% (w/w), relative to the total amount of the composition, from about 0.1% to about 2% (w/w), relative to the total amount of the composition, or any range or individual concentration encompassed by these example ranges.

In some embodiments, the compositions may further include an appetite suppressant such as, for example, 5HT (serotonin) transporter inhibitors, a NE (norepinephrine) transporter inhibitors, ghrelin antagonists, H3 (histamine H3) antagonist/inverse agonists, MCH1R (melanin concentrating hormone 1R) antagonists, a MCH2R (melanin concentrating hormone 2R) agonist/antagonists, NPY1 (neuropeptide Y Y1) antagonists, NPY5 (neuropeptide Y Y5) antagonists, NPY2 (neuropeptide Y Y2) agonists, NPY4 (neuropeptide Y Y4) agonists, mGluR5 (metabotropic glutamate subtype 5 receptor) antagonists, leptin, leptin derivatives, opioid antagonist, orexin antagonists, BRS3 (bombesin receptor subtype 3) agonists, CCK-A (cholecystokinin-A) agonists, CNTF (ciliary neurotrophic factors), CNTF derivatives, 5HT2c (serotonin receptor 2c) agonists, Mc4r (melanocortin-4 receptor) agonists, monoamine reuptake inhibitors, serotonin reuptake inhibitors, GLP-1 (glucagon-like peptide 1) agonists, topiramates, phytopharm compound 57, and the like and pharmaceutically acceptable salts and esters and combinations thereof. Such appetite suppressant may be In certain embodiments, the appetite suppressant may be an extract from, for example, grapes, pine, cocoa, tamarind, tomato, peanut, almond, apple, cranberry and blueberry. Such extract may include about 50 mg to about 3 gram procyanidins, which may act as an appetite suppressant.

The amount of appetite suppressant in the compositions described above may be from about 0.05% to about 20% (wt/wt), relative to the total amount of the composition or in some embodiments, from about 0.1% to about 10% (wt/wt), relative to the total weight of the composition, from about 0.1% to about 5% (w/w), relative to the total amount of the composition, from about 0.1% to about 2% (w/w), relative to the total amount of the composition, or any range or individual concentration encompassed by these example ranges.

In some embodiments, the compositions may further include an anti-inflammatory compound such as methotrexate, tofacitinib, 6-mercaptopurine, azathioprine sulphasalazine, mesalazine, olsalazine chloroquinine/hydroxychloroquinine, penicillamine, aurothiomalate (intramuscular and oral), azathioprine, cochicine, corticosteroids (oral, inhaled, and local injection), a beta-2 adrenoreceptor agonist (salbutamol, terbutaline, salmeteral), xanthine (theophylline, aminophylline), cromoglycate, nedocromil, ketotifen, ipratropium and oxitropium, cyclosporin, FK506, rapamycin, mycophenolate mofetil, leflunomide, an NSAID (e.g. ibuprofen), a corticosteroid (e.g. prednisolone), a phosphodiesterase inhibitor, an adensosine agonist, an antithrombotic agent, a complement inhibitor, an adrenergic agent, an agent that interferes with signalling by proinflammatory cytokines such as TNF or IL-1 (e.g., a NIK, IKK, p38 or MAP kinase inhibitor), an IL-1 converting enzyme inhibitor, a T-cell signalling inhibitor (e.g. a kinase inhibitor), a metalloproteinase inhibitor, sulfasalazine, a 6-mercaptopurine, an angiotensin converting enzyme inhibitor, a soluble cytokine receptor (e.g. soluble p55 or p75 TNF receptors and the derivatives p75TNFRigG (etanercept) and p55TNFRigG (Lenercept), siL-1RI, siL-1RII, siL-6R), an antiinflammatory cytokine (e.g. IL-4, IL-10, IL-11, IL-13 and TGF), celecoxib, folic acid, hydroxychloroquine sulfate, rofecoxib, etanercept, infliximab, adalimumab, certolizumab, tocilizumab, abatacept, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, cortisone, betamethasone, morphine sulfate, lidocaine hydrochloride, indomethacin, glucosamine sulf/chondroitin, amitriptyline HCl, sulfadiazine, oxycodone HCV acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL1S, BIRB-796, SCIO-469, VX-702, AMG-548, VX-740, Roflumilast, IC-485, CDC-801, S1PI agonists (such as FTY720), a PKC family inhibitor (e.g. Ruboxistaurin or AEB-071) or Mesopram, budenoside; epidermal growth factor; a corticosteroid; cyclosporin, sulfasalazine; an aminosalicylate; 6-mercaptopurine; azathioprine; metronidazole; a lipoxygenase inhibitor; mesalamine; olsalazine; balsalazide; an antioxidant; a thromboxane inhibitor; an IL-1 receptor antagonist; an anti-IL-1 monoclonal antibody; an anti-IL-6 monoclonal antibody; a growth factor; an elastase inhibitor; a pyridinyl-imidazole compound; an antibody to or antagonist of other human cytokines or growth factors (e.g. TNF, LT, IL-1, IL-2, IL-6, IL-7, IL-8, IL-12, IL-15, IL-16, IL-23, EMAP-II, GM-CSF, FGF, and PDGF); a cell surface molecule (e.g. CD2, CD3, CD4, CD8, CD25, CD28, CD30, CD40, CD45, CD69, or CD90 or their ligands); methotrexate; cyclosporine; FK506; rapamycin; mycophenolate mofetil; leflunomide; an NSAID (e.g. ibuprofen); a corticosteroid (e.g. prednisolone); a phosphodiesterase inhibitor; an adenosine agonist; an antithrombotic agent; a complement inhibitor; an adrenergic agent; an agent that interferes with signalling by proinflammatory cytokines such as TNF 5 or IL-1 (e.g. a NIK, IKK, or MAP kinase inhibitor); an IL-1 converting enzyme inhibitor; a TNF converting enzyme inhibitor; a T-cell signalling inhibitor such as kinase inhibitors; a metalloproteinase inhibitor; sulfasalazine; azathioprine; a 6-mercaptopurine; an angiotensin converting enzyme inhibitor; a soluble cytokine receptor (e.g. soluble p55 or p75 TNF receptors, siL-1RI, siL-1RII, siL-6R), an antiinflammatory cytokine (e.g. IL-4, IL-10, IL-11, IL-13 or TGF), therapeutic agents that target an intrinsic checkpoint blockade, such as, for example, the gene encoding Cytokine-inducible SH₂-containing protein (CISH), antibody BGB-A317, Nivolumab, or Pembrolizumab, atezolizumab, avelumab, durvalumab, ipilimumab, and the like and combinations thereof. The amount of anti-inflammatory in the composition may be from about 0.01% to about 5% (w/w), relative to the total amount of the composition, or in some embodiments, from about 0.1% to about 1% (w/w), relative to the total amount of the composition, or any range or individual concentration encompassed by these example ranges.

The oils and other active agents discussed above can be supplemented with any of the additives discussed below and can be incorporated into tinctures, orals, pills, tablets, capsules, lip balms, and the like as discussed below. Thus, the form of the compositions of the invention is not limited.

Another embodiment of the present invention is a method of making the composition in the form of a tincture. Tinctures are herbal extracts that provide a method for oral administration of an herbal component of components to a subject in need of treatment. Tinctures are prepared by mixing an herb or herbs or components or combinations thereof with a suitable solvent wherein a component or components of an herb or combinations thereof are extracted into a solvent in which the component or components of the herbs are reasonably soluble. Suitable tincture solvents in the present invention include pharmacologically acceptable solvent such as organic solvents, water based solvents, alcohols, and other orally administrable solvents such as, but not limited to, water, purified water, preserved water, vegetable glycerin, propylene carbonate, 3-methoxy-3-methyl-1-butanol (MMB), polyethylene glycerol, rice bran oil, and combinations thereof.

In some embodiments, the composition can be in the form of a tonic. Tonics are extracts that provide a method for oral administration of an herbal component or components to a subject in need of treatment. Tonics are prepared by mixing an herb or herbs or components or combinations thereof with a suitable solvent wherein a component or components of an herb or herbs or combinations thereof are extracted into a solvent by aid of heating, often heat necessary such that the solvent reaches its boiling temperature, in which the component or components of the herb are reasonably soluble. Suitable tonic solvents in the present invention include pharmacologically acceptable solvents such as organic solvents, water based solvents, alcohols, and other orally administrable solvents such as, but not limited to, water, purified water, preserved water, vegetable glycerin, propylene carbonate, 3-methoxy-3methyl-1-butanol (MMB), polyethylene glycol, rice bran oil, and combinations thereof.

In some embodiments, the composition can be in the form of a tablet. Tablets are pharmaceutical oral doses of a medicament or medicaments that are formed by molding or compression. Such embodiments are comprised of the medicament or medicaments and may be further comprised of suitable excipients such as, but not limited to, diluents, binders, granulating agents, gildants, lubricants, disintegrants, sweeteners, and pigments. Tablets in the present invention may also be coated with a pigment to increase the visual appearance of the tablet, to increase the identifiability of the tablet, to increase the ease with which the tablet is orally administered, to make the tablet more easily swallowed, to control the release of the medicament or medicaments, or to make the tablet more resistant to environmental degradation factors, or a combination or combinations thereof.

In another embodiment, the composition can be in the form of a capsule. Capsules generally fall within the class of either hard-shelled capsules or soft-shelled capsules, but need not be restricted to either class. Hard shelled capsules generally, but need not necessarily, contain dry, powdered, or granular components while soft-shelled capsules primarily, but need not necessarily, contain oils or medicaments or combinations thereof.

Another embodiment of the present invention is directed to methods for treating obesity, eating disorders, metabolic disorders, and other body weight related diseases and disorders, which includes administering a therapeutically effective amount of the compositions described above. Administering can be carried out by various means including oral and topical administration. Administering can be carried out for various time periods from one week to two or more years or until the desired effect has been achieved. Administration can be carried out one, two, three, or four times per day, and in some embodiments, administration can be carried out at meals.

Dosing may vary depending on various physiological properties of the individual subject, such as, weight, height, age, and the like or other properties known to the person of ordinary skill in the art. For example, the compositions of embodiments may administered at a daily dosage of about 0.0001 mg/kg to about 100 mg/kg of body weight, about 0.001 mg/kg to about 50 mg/kg of body weight, and any range or individual concentration encompasses by these ranges. The compositions may be given in a single dose or in divided doses two to six times per day, or in sustained release form.

In some embodiments, the method may include tapering doses of cannabidiol. Thus, embodiments may include a treatment regimen in which compositions containing different concentrations of cannabidiol are administered over the course of treatment. For example, some embodiments include applying one or more doses of a first composition containing greater than 10% (w/w) cannabidiol to a subject in need of treatment then subsequently administering one or more doses of a second composition containing less cannabidiol than the first topical composition to the subject in need of treatment. For example, if the first composition contains about 20% (w/w) cannabidiol, the second composition may include 19% (w/w) or less cannabidiol. In further embodiments, the method may include administering one or more doses of a third composition containing less cannabidiol than the second composition, a fourth composition containing less cannabidiol than the third composition, and so on. In particular embodiments, the method may include administering a composition containing a maintenance dose of cannabidiol that is equal to or less than the final dosage of a dosage regimen containing two or more compositions of decreasing dosage. The maintenance dose may provide sufficient cannabidiol to reduce or eliminate potential recurrence of the dermatological disease.

1. A composition comprising a cannabinoid, bioenhancer, and a pharmaceutically acceptable carrier, excipient, diluent, reagent, or combinations thereof.

2. The composition of claim 1, wherein the cannabinoid has a concentration of about 20% (w/w) to about 1% (w/w) cannabinoid, relative to a total amount of the composition.

3. The composition of claim 1, wherein the cannabinoid is selected from the group consisting of cannabidiol, cannabidiol isomer, cannabidiol analog, or combinations thereof.

4. The composition of claim 1, wherein the bioenhancer has a concentration of about 0.05% (w/w) to about 20% (w/w), relative to a total weight of the composition.

5. The composition of claim 1, wherein the bioenhancer is a cyclic peptide.

6. The composition of claim 1, wherein the bioenhancer is piperidine or a piperidine derivative.

7. The composition of claim 1, further comprising an appetite suppressant.

8. The composition of claim 7, wherein the appetite suppressant has a concentration of about 0.05% (w/w) to about 20% (w/w), relative to the total amount of the composition.

9. The composition of claim 7, wherein the appetite suppressant is an extract selected from the group consisting of grapes, pine, cocoa, tamarind, tomato, peanut, almond, apple, cranberry and blueberry.

10. A method for reducing the weight of a mammal comprising administering to the mammal a composition comprising a cannabinoid, bioenhancer, and a pharmaceutically acceptable carrier, excipient, diluent, reagent, or combinations thereof.

11. The method of claim 10, wherein the cannabinoid has a concentration of about 20% (w/w) to about 1% (w/w) cannabinoid, relative to a total amount of the composition.

12. The method of claim 10, wherein the cannabinoid is selected from the group consisting of cannabidiol, cannabidiol isomer, cannabidiol analog, or combinations thereof.

13. The method of claim 10, wherein the bioenhancer has a concentration of about 0.05% (w/w) to about 20% (w/w), relative to a total weight of the composition.

14. The method of claim 10, wherein the bioenhancer is a cyclic peptide.

15. The method of claim 10, wherein the bioenhancer is piperidine or a piperidine derivative.

16. The method of claim 10, wherein the composition further comprises an appetite suppressant.

17. The method of claim 16, wherein the appetite suppressant has a concentration of about 0.05% (w/w) to about 20% (w/w), relative to the total amount of the composition.

18. The method of claim 16, wherein the appetite suppressant is an extract selected from the group consisting of grapes, pine, cocoa, tamarind, tomato, peanut, almond, apple, cranberry and blueberry.

19. The method of claim 10, wherein the mammal is overweight, obese, has an eating disorder, has a metabolic disorder, or combinations thereof.

20. The method of claim 10, wherein the mammal is a human.
